(19) United States
(12) Patent Application Publication
(10) Pub. No.: US 2013/0131546 A1
(43) Pub. Date: May 23, 2013

(54) SYSTEM FOR COLLECTING AND PROCESSING BONE MARROW

(71) Applicants: Ali Faghani AZIMPOOR, Houston, TX (US); Lou BLASETTI, Quincy, MA (US); Mark KIBBE, Carver, MA (US)

(72) Inventors: Ali Faghani AZIMPOOR, Houston, TX (US); Lou BLASETTI, Quincy, MA (US); Mark KIBBE, Carver, MA (US)

(73) Assignee: HARVEST TECHNOLOGIES CORPORATION, Plymouth, MA (US)

(21) Appl. No.: 13/303,407

(22) Filed: Nov. 23, 2011

(51) Int. Cl. A61B 10/02

(52) U.S. Cl. 600/566

(57) ABSTRACT

Methods and systems for collecting and processing bone marrow in a closed system are provided. The systems and methods include the use of a bone marrow aspiration needle and guide configured to puncture cortical bone. Tube assembly is in fluid flow communication with the bone marrow aspiration needle. A first syringe is in fluid flow communication with the tube assembly and the bone aspiration. A bone marrow aspirate collection container in fluid flow communication with the first syringe and the tube assembly. A second syringe is connected to the collection container, a bone marrow aspirate filter in fluid flow communication with the tube assembly to filter bone marrow aspirate from the first syringe into the collection container. At least one flow controller is configured to direct the flow of bone marrow aspirate between the aspiration needle, the first syringe and the collection container.

### TECHNICAL FIELD

The present invention generally relates to a method and system for facilitating the collection and processing of biological material from a patient. More particularly, the present invention relates to systems and methods for collecting and processing bone marrow.

### BACKGROUND OF THE INVENTION

In general, bone marrow is aspirated using a bone marrow aspiration needle. Such needles include a hollow body member, in which a stylet is inserted and used to puncture the outer cortical bone of a patient. Once the cortical bone has been penetrated, the stylet is removed and a needle is inserted in its place. Then the desired bone marrow is aspirated via the needle. Typically, prior art methods of bone marrow collection involve the use of a syringe functionally attached to a bone marrow aspiration needle. Bone marrow is withdrawn into the syringe and the syringe is then disconnected from the aspiration needle and either used in a procedure directly, or connected to an external device for concentration or storage. For many procedures, the amount of bone marrow collected cannot be extracted in a single syringe draw.

Many prior art bone marrow collection systems are open systems, which allow the bone marrow to be exposed to the environment, and thus subject to contamination. Generally, such systems require multiple connections or disconnections between collection and processing components, including the aspiration needle, in order to collect enough bone marrow to be useful for a particular application or to collect enough bone marrow for concentration and later use. Each connection or disconnection is an opportunity for contamination and loss of valuable bone marrow aspirate. Also, undesired tissue can sometimes clog or otherwise obstruct parts of the collection system.

Thus, a need exists for a bone marrow collection system that minimizes potential for exposure of the bone marrow aspirate to the outside environment and contamination, and allows for the collection of relatively large significant amounts of bone marrow.

### SUMMARY OF THE INVENTION

In a first aspect, the invention includes a system for collecting and processing bone marrow. The system includes a first syringe, a bone marrow aspirate collection container, a tube assembly in fluid flow communication with the bone marrow aspiration needle, the first syringe and the collection container. The bone marrow aspiration needle includes a guide configured to puncture cortical bone and may be hollow to allow for the aspiration of bone marrow therethrough. The tube assembly allows the bone marrow aspirate to flow from the bone marrow aspiration needle to the first syringe and from the first syringe to the collection container. A second syringe is connected to the collection container to allow the bone marrow aspirate to be removed from the collection container. A bone marrow aspirate filter is located between the bone marrow aspiration needle and the second syringe to filter bone marrow aspirate. One or more flow controllers, such as clamps, are located in the system direct the flow of bone marrow aspirate between the aspiration needle, the first syringe and the collection container.

The present invention also includes a method for facilitating the collection and processing of bone marrow. The method includes providing a bone marrow aspiration needle and connecting a tube assembly in fluid flow communication with the bone marrow aspiration needle. A first syringe is connected in fluid flow communication with the tube assembly and the bone marrow aspiration needle to allow bone marrow aspirate to flow from the bone marrow aspiration needle tube into the first syringe. A bone marrow aspirate collection container is connected in fluid flow communication with the first syringe and the tube assembly to allow the bone marrow aspirate to flow from the first syringe into the collection container. A second syringe is connected to the collection container to allow the bone marrow aspirate to be removed from the collection container into the second syringe. A bone marrow aspirate filter is located between the bone marrow aspiration needle and the second syringe to filter bone marrow aspirate from the bone marrow aspiration needle into the second syringe. One or more flow controllers, such as clamps, configured to direct the flow of bone marrow aspirate.

The invention further includes a method for collecting and processing bone marrow. The method includes using a bone marrow aspiration needle and guide to puncture cortical bone, and aspirating bone marrow through a hollow portion of the bone marrow aspiration needle, needle, flowing the bone marrow aspirate from the bone marrow aspiration needle through the tube assembly and into a first syringe, flowing the bone marrow aspirate from the first syringe into a collection container removing the bone marrow aspirate from the collection container into a second syringe connected to the collection container, filtering the bone marrow aspirate from the first syringe using a bone marrow aspirate filter and controlling the flow of bone marrow aspirate between the aspiration needle, the first syringe and the collection container using one or more flow controllers.

These, and other objects, features and advantages of this invention will become apparent from the following detailed description of the various aspects of the invention taken in conjunction with the accompanying drawings.

### BRIEF DESCRIPTION OF THE DRAWINGS

FIG. 1 shows an example embodiment of a system according to the present invention.

FIG. 2 depicts a conceptual flow diagram of one embodiment according to one aspect of the invention.

### DETAILED DESCRIPTION OF THE INVENTION

The invention includes a system for collecting and processing bone marrow including a bone marrow aspiration needle and guide configured to puncture cortical bone, the needle being hollow to allow for the aspiration of bone marrow therethrough. The system also includes a first syringe, a bone marrow aspirate collection container, a tube assembly, to allow bone marrow aspirate to flow from the bone marrow aspiration needle to the first syringe and from the first syringe to the collection container. A second syringe is connected to the collection container to allow the bone marrow aspirate to be removed from the collection container into the second syringe. A bone marrow aspirate filters bone marrow from the bone marrow aspiration needle prior to transfer into the second syringe. One or more flow controllers, such as clamps, are used to direct the flow of bone marrow aspirate between the aspiration needle, the first syringe and the collection container.

The system is a closed system wherein bone marrow aspirate from the bone marrow aspiration needle is isolated from the environment when processed through the system including the tube assembly, collection container, syringes and filter. Contaminants are prevented from entering the system from the air or environment after the duct assembly is connected to the aspiration needle. Also, connections of various components of the system such as, for example, the second syringe to the bone marrow aspirate filter are sterile connections, as are well known in the art.

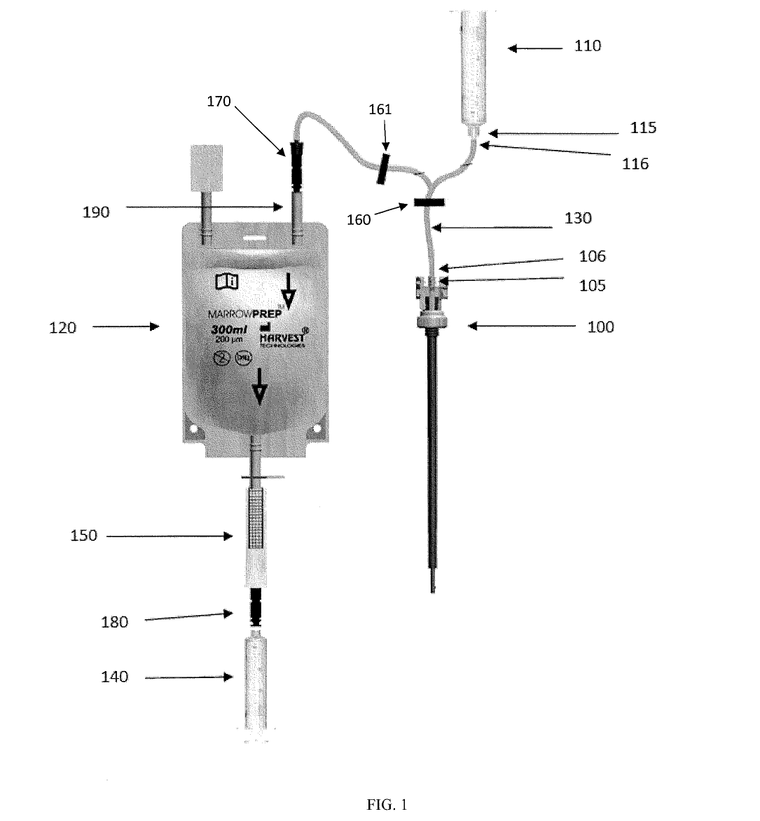

As shown in FIG. 1, a bone marrow aspiration needle and guide (hereinafter “bone marrow aspiration needle”) (100) is connected in fluid-flow communication with a tube assembly (130). The tube assembly (130) is also connected in fluid flow communication with a first syringe (110) and a bone marrow aspirate collection container (120). The tube assembly (130) is connected to the bone marrow aspiration needle (100), first syringe (110), and bone marrow aspirate collection container (120), for example, by using luer lock or similar sterile connections. As shown in FIG. 1, the invention wherein the bone marrow aspiration needle (100) has a female luer connection (105) connecting to a male luer connection (106) on the tube assembly (130). FIG. 1 also shows the first syringe (110) having a male luer connection (115), connecting to a female luer connection (116) on the tube assembly (130). Also, the bone marrow aspirate collection container (120) is connected to the tube assembly (130). For example, the container (120) may have a female luer connection or aseptic spike (190) that connects to a male luer connection or port (170) on the duct assembly (130). The bone marrow aspirate collection container (120) is also in fluid flow communication with a second syringe (140). The bone marrow aspirate collection container (120) and the second syringe (140) may also be connected using a luer lock or other aseptic connections (180).

FIG. 1 shows a bone marrow aspirate filter in direct connection with the second syringe (140) and connected to the second syringe (140) via a luer lock connection (180). A bone marrow aspirate filter (150) may be located between the bone marrow aspirate collection container (120) and the second syringe (140), and may be attached to each in a variety of ways. For example, the filter (150) may be integrally formed to a port at the end or bottom of the container (120). Also, the second syringe may be connected to the filter (150) using a leur lock connection (180) or other aseptic means. Alternatively, the bone marrow aspirate filter (150) marrow aspirate filters (150), which are capable of filtering undesirable materials from the bone marrow aspirate may be used. The bone marrow aspirate filter (150) may include, but are not limited to, screens, nets, densely packed material, for example, beads, or any other application-appropriate barrier that excludes undesired material from the bone marrow aspirate while allowed the desired aspirate fraction to pass through the barrier.

FIG. 1 also shows one or more flow controllers, for example, clamps (160 and 161), that are operably connected to the duct assembly (130). Each flow controller, including clamps (160 and 161) may have an “open” position and a “closed” position the “open” position allows bone marrow aspirate to flow through the particular portion of the tube assembly (130). The “closed” position prevents the flow of bone marrow through the particular portion of the tube assembly (130). Embodiments of the invention may have only one flow controller or several, depending upon the specific embodiment of the present invention that is being used. Several forms of flow controllers other than clamps (160 and 161) may be used including valves and/or stopcocks.

As shown in FIG. 1, clamps 160 and 161 are used. Clamp (160) would be set to the “open” position and clamp (161) set to the “closed” position in order to draw bone marrow from a patient, through the bone marrow aspiration needle (100), into a portion of the duct assembly (130) and into the first syringe (110). Once the bone marrow aspirate is drawn into the first syringe (110), clamp (160) is closed clamp (161) opened so that the bone marrow aspirate may flow from the first syringe (110), through a portion of the tube assembly (130), and into the bone marrow aspirate collection container (120).

The bone marrow aspiration needle (100) may be of any suitable form to extract bone marrow from the patient's bone. An example of a bone marrow aspiration needle that is suitable for use in the present invention is found in U.S. patent application Ser. No. 13/303,345 filed on Nov. 23, 2011 by Azimpoor et al. entitled Bone Aspiration Device and Needle, which is hereby incorporated by reference in its entirety.

The tube assembly (130) may be made of a variety of materials such as a flexible plastic or rubber tubing. Many embodiments of this aspect of the present invention will employ flexible tubing may be used for the tube assembly.

The collection container (120) may vary according to aspects of the present invention. Exemplary bone marrow aspirate collection containers (120) include, for example, flexible bags such as blood bags as are well known in the art. Such bags are sterilizable prior to use. In certain embodiments, the collection container may have patient identifying information on it. Additionally, the collection container may be of a variety of sizes and capacities, depending upon the amount of bone marrow aspirate required and/or the amount or nature of any additives that may be present for a particular procedure. Preferably, the second syringe is connected to the bone marrow aspirate collection container opposite the end where the first syringe is operably connected. This facilitates the transfer of aspirate from the container to the second syringe without turning the container upside down. With this configuration the container may be hung so that gravity may assist the flow of aspirate from connection (190) to second syringe (140).

Syringes suitable for use according to this aspect of the invention may be any variety of syringes capable of being connected to the systems of the present invention in a closed, or sterile, manner. The specific size and storage capacity of each syringe may vary in an application dependent manner, depending upon how much bone marrow is required for a particular application of the present invention.

The connections between the syringes, container, filter, tube assembly and aspiration needle are preferably sterile connections, but various connections may be used including spikable, luer lock and luer slip connections. The sterile connections maintain a sterile environment for the bone marrow aspirate as it flows through the various aspects of the present invention, without exposing the aspirate to the outside environment. Also each of these components may be sterilized prior to connection with other components of this aspect of the invention, or the entire system may be sterilized after assembly is complete, so long as the assembled systems are sterile or otherwise suitable to collect and store medically usable bone marrow.

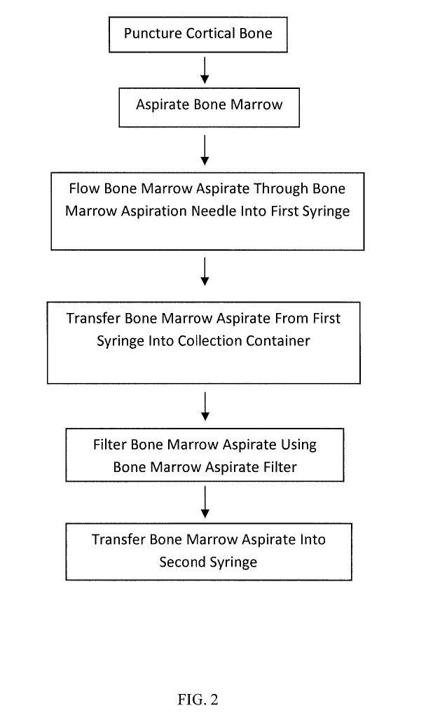

The present invention also includes a method for facilitating the collection and processing of bone marrow. FIG. 2 shows a conceptual flow diagram of a process for collecting this exemplary embodiment, and referring to FIGS. 1 and 2, a bone marrow aspiration needle (100) is used to facilitate penetration of cortical bone to access to the patient's bone marrow. A tube assembly (130) is connected to both a first syringe (110) and the bone marrow aspiration needle (100). A bone marrow aspirate collection container (120) is also connected to the tube assembly (130) as described above. A bone marrow aspirate filter (150), which is preferably pre-connected, to the tube assembly (130) or to a second syringe (140). The bone marrow aspirate filter (150) may be located such that the bone marrow aspirate is filtered before it enters the bone marrow aspirate collection container (120), or such that it is filtered after the bone marrow aspirate leaves the bone marrow aspirate collection container (120), for example, to enter the second syringe (140). In the embodiment shown in FIG. 2, if the bone marrow aspirate filter (150) is connected to the duct assembly (130), then the second syringe (140) is connected to the bone marrow aspirate collection container (120). If, instead, the bone marrow aspirate filter (150) is connected to the second syringe (140), then the bone marrow aspirate filter (150) is connected to the bone marrow aspirate collection container. Other embodiments may have alternative arrangements of these components and such varying arrangements are contemplated as within the scope of the present invention.

FIG. 2 also shows that one or more flow controllers such as clamps or stopcocks are provided in order to direct the flow of bone marrow aspirate between components of this aspect of the invention.

The invention includes a method for collecting and processing bone marrow, the method including the steps of using a bone marrow aspiration needle and guide configured to puncture cortical bone. Bone marrow is aspirated through a hollow portion of the bone marrow aspiration needle (100). Typically, a stylet is inserted into the bone marrow aspiration needle (100), and a hammer is used to drive the bone marrow aspiration needle through the cortical bone. The tube assembly (130) is connected in fluid flow communication with the bone marrow aspiration needle (100). Bone marrow aspirate is flowed from the bone marrow aspiration needle (100) through the tube assembly (130) and into a first syringe (110), which in fluid flow communication with the tube assembly (130) and the bone marrow aspiration needle (100). The first syringe (110) may include anticoagulant therein to prevent coagulation of the bone marrow therein. The bone marrow aspirate is then flowed from the first syringe (110) into a collection container (120), which is in fluid flow communication with the first syringe and the tube assembly (130). The collection container may also contain anticoagulant therein, if desired. The bone marrow aspirate is removed from the collection container (120) into a second syringe (140), which is connected to the collection container. The bone marrow aspirate is filtered using a bone marrow aspirate filter (150), within the tube assembly and located between the collection container (120) and the second syringe (140). The flow control clamps (160 and 161) are manipulated to allow bone marrow to flow from the bone marrow aspiration needle (100) through the tube assembly (130) and into the first syringe (110) without also flowing toward bone marrow collection container (120).

Certain steps may vary according to where the bone marrow aspirate filter is physically located within the system used to practice the present method. According to the alternative method, after the bone marrow aspirate is made to flow into a first syringe (110), the bone marrow aspirate may be made to flow into the bone marrow collection container (120), then be filtered by the bone marrow aspirate filter (150), and finally caused to flow into a second syringe (140). Alternatively, the bone marrow aspirate may be filtered through the bone marrow aspirate filter, then transferred into the bone marrow aspirate collection container, and finally into the second syringe. In this case, the flow control clamps are manipulated so that bone marrow aspirate flows from the first syringe through tube assembly, through bone marrow aspirate filter and into the bone marrow collection container without also flowing through the tube assembly into the bone marrow aspiration needle. Transfer of the bone marrow aspirate collection container to the second syringe may be provided by a user drawing back the plunger of second syringe.

The system of the present invention is operable as a closed system wherein the bone marrow aspirate is not exposed to the environment after it flows from the aspirate needle or once the duct assembly is corrected to the aspiration needle. The duct assembly, first syringe, flow controllers, bone marrow aspirate collection container, bone marrow aspirate filter, and second syringe may be pre-connected prior to use of the system. In this regard, only the connection of the duct assembly to the aspirate needle need be performed by the user. And, this connection may be facilitated using sterile or closed connection devices or techniques. Alternatively, some components of the system may not be pre-connected, such as for example, the second syringe and the bone marrow aspirate collection container and/or for example, the bone marrow aspirate collection container and the duct assembly. If such components are not pre-connected, they may be connected by the user of the system during the collection process. In such a situation, sterile or closed connection techniques should be utilized to prevent contamination of the bone marrow and its exposure to the environment.

Aspects of the invention are suitable for use in a variety of bone marrow processing applications, including use in removal and processing of vertebral bone marrow.

While several aspects of the present invention have been described and depicted herein, alternative aspects may be affected by those skilled in the art to accomplish the same objectives. Accordingly, it is intended by the appended claims to cover all such alternative aspects as fall within the true spirit and scope of the invention.

1. A system for collecting and processing bone marrow comprising:
a bone marrow aspiration needle and guide configured to puncture cortical bone, said needle being hollow to allow for the aspiration of bone marrow therethrough;
a first syringe;
a bone marrow aspirate collection container;
a tube assembly connectable in fluid flow communication with said bone marrow aspiration needle, said first syringe and said collection container to allow bone marrow aspirate to flow from said bone marrow aspiration needle to said first syringe and from said first syringe to said collection container;
a second syringe connected to said collection container to allow said bone marrow aspirate to be removed from said collection container into said second syringe;
a bone marrow aspirate filter in fluid flow communication with said tube assembly and located between said bone marrow aspiration needle and said second syringe to filter bone marrow aspirate from said bone marrow aspiration needle into said second syringe; and
one or more flow controllers configured to direct the flow of bone marrow aspirate between said aspiration needle, said first syringe and said collection container.

2. The system of claim 1 wherein said tube assembly comprises at least three branches wherein a first branch is connected to said bone aspiration needle, a second branch connected to said first syringe, and a third branch connected to said collection container.

3. The system of claim 2 wherein said flow controller is configured to selectively prevent flow of bone marrow aspirate into at least one of said first, second and third branches of said tube assembly.

4. The system of claim 1 wherein said flow controller comprises at least one clamp or at least one valve.

5. The system of claim 1 wherein said tube assembly is connected to a first end of said collection container and said second syringe is connected to a second end of said collection container.

6. The system of claim 5 wherein said first end of said collection container is opposite said second end.

7. The system of claim 1 wherein said bone aspiration needle, said tube assembly, said first syringe, said collection container, said bone marrow aspirate filter, and said second syringe are connected to form a closed system wherein bone marrow aspirate from said bone marrow aspiration needle therein is isolated from the environment.

8. A method for facilitating the collecting and processing of bone marrow comprising:
providing a bone marrow aspiration needle and guide configured to puncture cortical bone, said needle being hollow to allow for the aspiration of bone marrow therethrough;
providing a tube assembly in fluid flow communication with said bone marrow aspiration needle;
connecting a first syringe in fluid flow communication with said tube assembly and said bone marrow aspiration needle to allow bone marrow aspirate to flow from said bone marrow aspiration needle through said duct assembly and into said first syringe;
connecting a bone marrow aspirate collection container in fluid flow communication with said first syringe and said duct assembly to allow said bone marrow aspirate to flow from said first syringe into said collection container;
connecting a second syringe to said collection container to allow said bone marrow aspirate to be removed from said collection container into said second syringe;
connecting a bone marrow aspirate filter in fluid flow communication with said tube assembly and located between said bone marrow aspiration needle and said second syringe to filter bone marrow aspirate from said bone marrow aspiration needle into said second syringe; and
providing one or more flow controllers to direct the flow of bone marrow aspirate between said aspiration needle, said first syringe and said collection container.

9. The method of claim 8 wherein said tube assembly comprises at least three branches wherein a first branch is connected to said bone aspiration needle, a second branch connected to said first syringe, and a third branch connected to said collection container.

10. The method of claim 9 wherein said flow controller is configured to selectively prevent flow of bone marrow aspirate into at least one of said first, second and third branches of said duct assembly.

11. The method of claim 8 wherein said flow controller comprises at least one clamp or at least one valve.

12. The method of claim 8 comprising connecting said tube assembly to a first end of said collection container and said second syringe is connected to a second end of said collection container, opposite said second end.

13. The method of claim 8 wherein said bone aspiration needle, said tube assembly, said first syringe, said collection container, said bone marrow aspirate filter, and said second syringe are connected to form a closed system wherein bone marrow aspirate from said bone marrow aspiration needle therein is isolated from the environment.

14. A method for collecting and processing bone marrow comprising:
using a bone marrow aspiration needle and guide to puncture cortical bone, and aspirating bone marrow through a hollow portion of said bone marrow aspiration needle;
connecting a tube assembly in fluid flow communication with said bone marrow aspiration needle;
flowing said bone marrow aspirate from said bone marrow aspiration needle through said tube assembly and into a first syringe in fluid flow communication with said tube assembly and said bone marrow aspiration needle;
flowing said bone marrow aspirate from said first syringe into a collection container in fluid flow communication with said first syringe and said tube assembly;
removing said bone marrow aspirate from said collection container into a second syringe connected to said collection container;
filtering said bone marrow aspirate from said first syringe into said collection container using a bone marrow aspirate filter in fluid flow communication with said tube assembly and located between said bone marrow aspiration needle and said second syringe; and
controlling the flow of bone marrow aspirate between said aspiration needle, said first syringe and said collection container using one or more flow controllers.

15. The method of claim 14 wherein said tube assembly comprises at least three branches wherein a first branch is connected to said bone aspiration needle, a second branch connected to said first syringe, and a third branch connected to said collection container.

16. The method of claim 15 wherein said flow controller is configured to selectively prevent flow of bone marrow aspirate into at least one of said first, second and third branches of said tube assembly.

17. The method of claim 14 wherein said flow controller comprises at least one clamp or at least one valve.

18. The method of claim 14 wherein said tube assembly is connected to a first end of said collection container and said second syringe is connected to a second end of said collection container, opposite said second end.

19. The method of claim 14 wherein said bone aspiration needle, said tube assembly, said first syringe, said collection container, said bone marrow aspirate filter, and said second syringe are connected to form a closed system wherein bone marrow aspirate from said bone marrow aspiration needle therein is isolated from the environment.

20. A kit for collecting and processing bone marrow comprising:
a bone marrow aspiration needle and guide configured to puncture cortical bone, said needle being hollow to allow for the aspiration of bone marrow therethrough;
a first syringe;
a bone marrow aspirate collection container;
a tube assembly connectable in fluid flow communication with said bone marrow aspiration needle, said first syringe and said collection container to allow bone marrow aspirate to flow from said bone marrow aspiration needle to said first syringe and from said first syringe to said collection container;
a second syringe connectable to said collection container to allow said bone marrow aspirate to be removed from said collection container into said second syringe;
a bone marrow aspirate filter in fluid flow communication with said tube assembly and located between said bone marrow aspiration needle and said second syringe to filter bone marrow aspirate from said bone marrow aspiration needle into said second syringe; and
one or more flow controllers configured to direct the flow of bone marrow aspirate between said aspiration needle, said first syringe and said collection container.

21. The kit of claim 20, wherein said duct assembly is connected to said collection container and said second syringe is connected to said container.

22. The kit of claim 20, wherein said one or more flow controllers is two flow controllers.

23. The kit of claim 20, wherein said bone marrow aspirate collection container comprises one connection on a first end of said collection container and a second connection on a second end of said collection container.

24. The kit of claim 23, wherein said first end of said collection container is opposite said second end of said collection container.
